(19) United States
(12) Patent Application Publication
(10) Pub. No.: US 2016/0143944 A1
(43) Pub. Date: May 26, 2016

(54) ANTIMICROBIAL COMPOSITIONS COMPRISING HYPOCHLOROUS ACID AND SILVER

(71) Applicant: PURICOE, INC., Malvern, PA (US)

(72) Inventors: Svetlana Panicheva, Malvern, PA (US); Mark N. Sampson, Malvern, PA (US); Ronan Stapleton, Malvern, PA (US)

(21) Appl. No.: 14/900,859

(22) PCT Filed: Jul. 1, 2014
(86) PCT No.: PCT/US14/45025

§ 371 Date: Dec. 22, 2015

Related U.S. Application Data
(60) Provisional application No. 61/841,616, filed on Jul. 1, 2013.

(51) Int. Cl. A61K 33/20; A61L 26/00; A61K 33/38

(52) U.S. Cl. CPC A61K 33/20; A61K 33/38; A61L 26/0004; A61L 26/0066; A61L 2300/404; A61L 2300/104; A61L 2300/106

(57) ABSTRACT

The present invention relates to methods and compositions for treating a surface characterized by microbial infection or colonization. Particularly, the methods of the present invention involve applying a hypohalous acid (e.g., hypochlorous acid) composition and a silver additive. The present invention is useful, for example, in disinfecting or cleaning a mammalian tissue, such as a wound or burn, or disinfecting or cleaning a hard surface, such as a medical device.

### PRIORITY

This application claims the benefit of, and priority to, U.S. Provisional Application No. 61/841,616, filed Jul. 1, 2013, which is hereby incorporated by reference in its entirety.

### FIELD OF THE INVENTION

The present invention relates to methods and compositions for treating a surface characterized by microbial infection or colonization. Specifically, microbial infection or colonization is reduced by administering or applying a hypohalous acid (e.g., hypochlorous acid) composition and a silver additive.

### BACKGROUND

Microbial infections or contaminations have a profound effect on human health and well-being. In particular, bacterial infections are known to cause a myriad of human illnesses, ranging from mild conditions such as ear infections, diarrhea, and skin disorders to life-threatening conditions such as bacterial meningitis which require immediate intervention. The elderly, children, hospitalized patients, and those with chronic diseases are especially at high risk for microbial infections.

Microorganisms can cause infections if they directly enter the body through cuts, open wounds, or other breaks in the skin. In addition, microorganisms can attach to non-living surfaces and form biofilms made up of extracellular polymers on, for example, medical devices. In this state, the microorganisms are tenaciously bound to the surface and are highly resistant to antimicrobial treatment. Increased use of intrusive devices (e.g., orthopedic devices, neurovascular shunts, prosthetic heart valves, cardiac pacemakers, contact lenses, intrauterine devices, vascular, peritoneal and urinary catheters, etc.) is paralleled by a growing risk of developing biofilm-related infections or contaminations associated with such devices. Further, treatment of microbial infections is hampered by the increasing emergence of antibiotic-resistant and/or novel virulent pathogens. Accordingly, there remains a need for improved methods and compositions for treating or preventing microbial infections or colonizations.

### SUMMARY OF THE INVENTION

In one aspect, the present invention provides a method for treating a surface characterized by microbial infection or colonization. The method involves applying a hypochlorous acid (HOCl) composition and a silver additive to the surface, thereby effectively reducing the microbial infection or colonization.

The invention is useful for treating surfaces affected by microbial infection or colonization. Such surfaces include a hard surface, including a tubing, contact lens, dental prosthesis, orthodontic device, surgical instrument, dental instrument, medical examination surface, bathroom surface, dental water line, fabric, or bandage or tissue dressing. Such surfaces can also include a medical device, such as a urinary catheter, mucous extraction catheter, suction catheter, umbilical cannula, intrauterine device, intravaginal device, intraintestinal device, endotracheal tube, bronchoscope, endoscope, electrodes, or external prosthesis. In addition, the surface can be a human or animal tissue or organ, such as a wound or burn. In an embodiment, the affected surface is characterized by bacterial biofilm formation.

In certain embodiments, the present invention involves treating infected or colonized surfaces. The bacterial infection can be a gram positive bacterial infection or a gram negative bacterial infection. The bacterial infection may include, for example, one or more of E. Coli, Salmonella sp., Shigella sp., Pseudomonas sp., Moraxella sp., Helicobacter sp., Legionella sp., Acinetobacter sp., Neisseria sp., Hemophilus influenzae, Klebsiella pneumoniae, Proteus mirabilis, Bacillus anthracis, Clostridium difficile spores and Enterobacter sp. In an embodiment, the surface is infected with Pseudomonas aeruginosa biofilm.

The method of the invention involves administering or applying a hypochlorous acid composition and a silver additive. The hypochlorous acid composition may be produced by electrolysis of saline. The hypochlorous acid composition may have a pH of from about 3.5 to about 7 and an available free chlorine (AFC) content of from about 20 parts per million (ppm) to about 2000 ppm. The silver additive may be one or more of elemental silver, silver salt, or ionic silver. In certain embodiments, the silver additive is silver nitrate, silver acetate, silver benzoate, silver carbonate, silver iodate, silver iodide, silver lactate, silver laurate, silver oxide, silver palmitate, and silver sulfadiazine. The silver additive can be applied as a solution or ointment having a concentration of at least about 5 ppm. In an embodiment, the silver additive is applied as a solution or ointment having a concentration of about 5 ppm to about 200 ppm. The hypochlorous acid and the silver additive can be applied as a single composition. Alternatively, the hypochlorous acid and the silver additive can be applied sequentially. In a further embodiment, the hypochlorous acid is applied as a liquid or hydrogel, whereas the silver additive is applied as a coating or additive on a wound dressing or medical device.

In another aspect, the present invention provides a kit for treating a surface for microbial contamination or infection. The kit includes a hypochlorous acid formulation and a silver additive. The kit may further include materials for wound dressing.

The hypochlorous acid composition may be produced by electrolysis of saline. In certain embodiments, the hypochlorous acid composition can have a pH of from about 3.5 to about 7, and an AFC of from about 20 ppm to about 2,000 ppm. The hypochlorous acid may be a liquid of hydrogel formulation.

In various embodiments, the silver additive may be a solution or an ointment. Alternatively, the silver additive can be a coating or a composition integral to a wound dressing or medical device, such as a urinary catheter, mucous extraction catheter, suction catheter, umbilical cannula, intrauterine device, intravaginal device, intraintestinal device, endotracheal tube, bronchoscope, endoscope, electrodes, or external prosthesis. The silver additive can be silver nitrate, silver acetate, silver benzoate, silver carbonate, silver iodate, silver iodide, silver lactate, silver laurate, silver oxide, silver palmitate, and/or silver sulfadiazine. The silver additive may be present at a concentration of at least 5 ppm. For example, the silver additive may be present at a concentration of about 5 ppm to about 200 ppm.

### BRIEF DESCRIPTION OF THE FIGURES

FIG. 1 shows a biofilm test study to determine the biocidal performance of various hypochlorous acid compositions with or without silver additive against Pseudomonas aeruginosa.

### DETAILED DESCRIPTION OF THE INVENTION

The present invention relates to methods and compositions for treating a surface characterized by microbial infection or colonization. Specifically, microbial infection or colonization is reduced by administering or applying a hypochlorous acid composition and a silver additive.

### Hypochlorous Acid, Solutions and Compositions

Hypochlorous acid (HOCl) is an oxidant and biocide that is produced by the human body's natural immune system. HOCl is generated as the final step of the Oxidative Burst Pathway, with large quantities of HOCl being released into phagocytic vesicles to destroy invading microorganisms. It is considered, without wishing to be bound by any theory, that hypochlorous acid exerts a biocidal effect by attacking the surface and plasma membrane proteins, impairing transport of solutes and the electrolyte balance of bacterial cells (Pieterson et al., Water S A, 22(1): 43-48 (1996)).

In accordance with the present invention, a composition comprising hypohalous acid (e.g., hypochlorous acid) is administered or applied to treat surfaces affected by microbial infection. The hypohalous acid composition is non-irritating and non-sensitizing to the skin, non-irritating to the eyes, not harmful if swallowed, show no evidence of mutagenic activity, and are safe for routine or prolonged use. An added advantage is that there is no resistance or tolerance developed by the microorganisms, as occurs with the use of conventional antibiotics, and there is generally no hypersensitivity as occurs with some agents conventionally administered to treat microbial infections.

The hypohalous acid solution may be generated electrochemically, such as by electrolysis of salt, such as saline (NaCl), and may contain a mixture of oxidizing species such as predominantly hypochlorous acid (HOCl) and sodium hypochlorite. Hypochlorous acid and hypochlorite are in equilibrium and the position of the equilibrium is determined solely by the pH, which may be controlled by the electrochemical generator. The hypohalous acid solution may have a pH of from about 2.5 to about 9, from about 3 to about 8, from about 3 to about 7.5, from about 3.5 to about 7, from about 4 to about 7, from about 4.0 to about 6.5, or from about 5.0 to about 6.0. For example, the hypohalous acid solution may have a pH of about 5.4. The pH of the solution can be controlled, for example, by modulating the chemical properties of the solution, or (where an electrolyzed solution is used as the source of hypohalous acid) the hydraulic regime within the electrochemical cell system, the applied electric current, or the recirculation of the catholyte produced by the electrochemical cell. Exemplary methods and apparatuses for preparing electrolyzed solutions of the present invention are disclosed in US Published Patent Application No. 2004/0060815, which is hereby incorporated by reference in its entirety. In other embodiments, the hypohalous acid solution may be produced chemically (e.g., by acidification of hypochlorite).

The hypohalous acid may be prepared by electrolysis of one or more halide salts, including CI, Br, and I. Thus, the hypohalous acid may include one or a mixture of HOCl, HOBr, and HOI. In certain embodiments, the electrolyzed solution is generated using a mixture of physiologically balanced salts, as disclosed in U.S. Pat. 6,426,066, which is hereby incorporated by reference in its entirety. Such salts may include sodium halides (e.g., NaCl), potassium halides (e.g., KCl) and magnesium halides (e.g., MgCl₂).

In certain embodiments, the electrolyzed solution consists of essentially hypohalous acid as the active agent (e.g., HOCl), but in certain other embodiments may contain, or may also contain, other oxidizing or radical producing species such as a hypohalite (e.g., hypochlorite), hydroxide, H₂O₂ and O₃. These species may provide additional biocidal activity, and may have additional benefits for clearing microbial debris, biofilm, or discharge. In certain embodiments, the hypohalous acid solution contains at least 80% hypohalous acid relative to the total concentration of hypohalous acid, hypohalite, and Cl₂ (as 100%). The hypohalous acid may have, however, at least 90%, at least 95%, or at least 98% hypohalous acid. Such embodiments may allow for higher levels of active chlorine to be administered, while avoiding any irritation as a result of the solution.

The biocidal activity of the hypohalous solution can be expressed in terms of available free chlorine or AFC. The hypohalous acid solution, such as a HOCl solution, may contain an AFC content of from about 2 to about 10,000 parts per million (ppm). In some embodiments, the solution of the invention has an AFC content of from about 2 to about 5,000 ppm, from about 2 to about 4,000 ppm, from about 2 to about 2,000 ppm, from about 2 to about 1,000 ppm, from about 20 to about 2,000 ppm, from about 20 to about 1,800 ppm, from about 20 to about 1,600 ppm, from about 20 to about 1,400 ppm, from about 20 to about 1,200 ppm, from about 20 to about 1,000 ppm, from about 20 to about 800 ppm, from about 20 to about 600 ppm, from about 20 to about 500 ppm, from about 20 to about 400 ppm, from about 20 to about 300 ppm, from about 20 to about 200 ppm, from about 20 to about 100 ppm, from about 20 to about 50 ppm, from about 50 to about 1,000 ppm, from about 50 to about 500 ppm, from about 50 to about 400 ppm, from about 50 to about 300 ppm, from about 50 to about 200 ppm, or from about 50 to about 100 ppm. The AFC level of the composition will depend upon the intended application. For example, in some embodiments involving application to human tissues or cells, the AFC may be in the range of from about 50 to about 500 ppm, such as from about 80 to about 400 ppm. While such solutions are potent biocides, such solutions are not generally irritating to the skin, eye, nasal mucosa, and ear, and are not harmful to medical devices.

The electrolyzed solution of the invention may also contain from about 0.1 to 2.0% w/v salt, such as NaCl. In some embodiments, the invention contains about 0.1 to about 1.5%, about 0.2 to about 1.5%, about 0.3 to about 1.5%, or about 0.4 to 1.5% w/v salt, or may be a normal saline solution (0.9% w/v NaCl). The hypohalous acid composition may be hypertonic, hypotonic, or isotonic with respect to physiological fluids, such blood, saliva or tears. In an embodiment, the hypohalous acid composition is either isotonic or hypotonic with respect to physiological fluids. While the hypohalous solution may be administered at room temperature, the solution may alternatively be heated, for example, to body temperature or about body temperature, or above body temperature. In certain embodiments, the hypohalous acid is administered at below body temperature.

In certain embodiments, the hypohalous acid solution may include a stabilizing amount of dissolved inorganic carbon (DIC), such as bicarbonate or carbonate of an alkali or alkaline earth metal (e.g., sodium, potassium, calcium, or magnesium). The DIC is incorporated at a “stabilizing amount,” which can be determined with reference to the change in the pH or AFC content of the solution over time. Generally, the solution is considered stabilized if the amount of AFC does not drop below about 75% of the initial value over a period of about 6 months. In certain embodiments, the AFC content is stabilized for at least one year from the production date of the solution. Further, the stability of the solution may be determined with reference to the pH. Generally, the solution is considered stabilized if the pH does not vary by more than 1 unit over a period of about 6 months. In certain embodiments, the pH is stabilized for at least one year from the production date of the solution. Exemplary hypohalous acid solutions including DIC and methods for preparing such solutions are disclosed in US Published Patent Application No. 2012/0237616, which is hereby incorporated by reference in its entirety.

The stabilizing amount of DIC (e.g., as added carbonate or bicarbonate) can be determined with reference to the AFC content. For example, in certain embodiments, the stabilizing amount of the carbonate or bicarbonate is incorporated into the solution at a molar ratio of from about 5:1 to 1:5 with respect to the AFC level. In some embodiments, the bicarbonates or carbonates are incorporated into the solution in at least equimolar amounts with respect to the AFC content (e.g., hypochlorous acid content). In still other embodiments, the DIC (e.g., bicarbonate or carbonate) is incorporated at about 5:1, about 2:1, about 1:1, about 1:2, about 1:3, or about 1:5 with respect to AFC content. For example, for solutions having an AFC content of from about 200 ppm to about 500 ppm, carbonate or bicarbonate may be incorporated at an amount of from about 300 mg/L to about 1500 mg/L to stabilize the solution. In certain embodiments, such solutions are stabilized by incorporating from about 400 to about 1000 mg/L of carbonate or bicarbonate.

The electrolyzed solution may have an oxidation reduction potential (redox) of greater than about +650 mV, greater than about +950 mV, such as about +1000 mV. A high redox potential allows for the quick and efficient destruction of microbes (bacteria, viruses, fungi and spores). Generally, the hypohalous acid is effective on a broad spectrum of bacterial, fungal, and/or viral pathogens.

### Silver Additives

In accordance with the present invention, a silver additive is used in conjunction with the hypohalous acid (e.g., hypochlorous acid) composition. Without wishing to be bound by any theory, it is believed that the combination of silver and, for example, hypochlorous acid, generates silver chloride and other species with enhanced and sustained antimicrobial activities.

As contemplated herein, the silver additive may be provided in various forms. In some embodiments, the silver additive is elemental silver. In some embodiments, the elemental silver can be prepared in the form of colloidal silver. The term “colloidal silver” as used herein relates to any preparation of elemental silver that is sufficiently finely dispersed to form a colloid solution when dispersed in water. The average silver particle size is generally in the range from 1 to 100 nanometers, typically 1 to 10 nanometers, corresponding to generally less than 10⁹ silver atoms per particle. Several different methods for the preparation of silver colloids are known in the art, including, but not limited to, mechanical milling, electrolytic processes, and chemical reduction of silver salts in solution, and any art-recognized method. The colloid can be provided in the form of a powder or of an aqueous dispersion (“colloid solution”). In some embodiments, colloidal silver can also contain a certain proportion of ionic silver in addition to elemental silver due to redox reactions on the surface of the silver particles. In other embodiments, the elemental silver can be prepared in the form of silver nanoparticles. As used herein, the term “silver nanoparticle” can include nanoparticles of silver metal, a silver metal alloy, oxidized silver or silver alloy or silver oxide. The term “silver metal” or “silver alloy” refers to those which have not yet been oxidized. In some embodiments, the “silver nanoparticles” can contain at least some silver oxide and are referred to herein as “silver oxide nanoparticles”. In such embodiments, the silver oxide nanoparticles can comprise a core of silver or silver alloy surrounded by a layer of the oxide. Alternatively, the silver oxide nanoparticles can consist entirely of silver oxide.

In some embodiments, the silver-containing compound can comprise a silver salt or ionic silver. The term “ionic silver” as used herein, can encompass both the cationic form of silver, Ag⁺, and the anionic silver thiosulfate complex, and other silver metal complexes. In particular embodiments, ionic silver is in the cationic form of silver, i.e., Ag⁺. The cationic form silver can be present in various silver salts. Examples of silver salts include, but are not limited to, silver nitrate, silver benzoate, silver carbonate, silver iodate, silver iodide, silver lactate, silver laurate, silver palmitate, silver oxide, silver bromide, silver fluoride, silver chloride, silver sulfate, silver dihydrogen citrate, silver alkylcarboxylate, silver sulphadiazine or silver arylsulfonate. Silver alkyl carboxylates are the silver salts of alkylcarboxylic acids preferably having from 1-12 aliphatic carbon atoms, or from 1-4 aliphatic carbon atoms, e.g., silver acetate. The aryl group of the arylsulfonate salts is an aromatic radical, e.g., optionally substituted phenyl or naphthyl, preferably alkaryl having 1 to 12 aliphatic carbon atoms, or alkylphenyl having from 1 to 4 aliphatic carbon atoms, e.g., p-toluenesulfonate.

In various embodiments, the present invention involves applying silver at a concentration of from about 5 ppm to about 1,000 ppm, from about 5 ppm to about 900 ppm, from about 5 ppm to about 800 ppm, from about 5 ppm to about 700 ppm, from about 5 ppm to about 600 ppm, from about 5 ppm to about 500 ppm, from about 5 ppm to about 400 ppm, from about 5 ppm to about 300 ppm, from about 5 ppm to about 200 ppm, from about 5 ppm to about 100 ppm, or from about 5 ppm to about 50 ppm. Such amounts can be delivered by in any appropriate form, such as by solution, ointment, or coating.

In some embodiments, the amount of the silver additive is present at a concentration of about 0.001 μM to about 100 μM, about 0.01 μM to about 80 μM, about 0.1 μM to about 50 μM, about 1 μM to about 30 μM, about 5 μM to about 25 μM, about 10 μM to about 25 μM, or about 15 μM to about 20 μM. In some embodiments, the amount of the silver additive is present at a concentration of about 1 μM to about 50 μM, about 1 μM to about 30 μM, about 5 μM to about 25 μM, about 5 μM to about 20 μM, about 10 μM to about 20 μM, or about 15 μM to about 20 μM. In some embodiments, the amount of the silver additive is present at less than 50 μM, at less than 25 μM, or less than 20 μM, or less than 10 μM.

### Compositions and Modes of Treatment

The present invention contemplates the application of a hypohalous acid (e.g., hypochlorous acid) composition and a silver additive. In an embodiment, the hypohalous acid composition and the silver additive are formulated together with one or more pharmaceutically acceptable carriers and/or diluents. In another embodiment, the hypohalous acid composition and the silver additive are each individually formulated or applied in the same or different pharmaceutically acceptable carriers and/or diluents.

The composition of the invention may be formulated as a liquid, such as an eye drop, eye wash, wash for contact lenses, gargle, nasal or throat spray, or ear drop. Alternatively, the composition may take the form of a cream, gel, and/or foam. Convenient applicators for creams, foams, and the like are known, and may be used in accordance with the present invention. Alternatively still, the composition of the invention may be formulated so as to be delivered by aerosol, mist, or steam. In an embodiment, the hypohalous acid (e.g., hypochlorous acid) composition is formulated as a liquid or hydrogel formulation. In another embodiment, the silver additive is formulated as a coating or a composition integral to a wound dressing or medical device. Methods for formulating silver as a coating or into wound dressing (e.g., gauze) are known in the art. For example, methods for incorporating silver into wound dressings are described in U.S. Pat. Nos. 4,728,323, 7,005,556, and 7,462,752, which are hereby incorporated by reference in their entireties. In a further embodiment, the silver additive is formulated as a solution or ointment. The hypohalous acid composition and the silver additive may be applied by the same or different route of administration.

The composition may further comprise a pharmaceutically acceptable carrier. Non-limiting examples of suitable carriers include polyvinyl alcohol, povidone, hydroxypropyl methyl cellulose, poloxamers, carboxymethyl cellulose, hydroxyethyl cellulose, and purified water. The compositions of the present invention may also include various other ingredients, such as tonicity agents, buffers, surfactants, co-solvents, viscosity building agents, preservatives, and other therapeutic agents.

Regarding tonicity agents, such agents may be employed to adjust the tonicity of a composition, for example, in the case of an ophthalmic composition, to the tonicity of natural tears. For example, sodium chloride, potassium chloride, magnesium chloride, calcium chloride, dextrose and/or mannitol may be added to the composition to approximate physiological tonicity. Such an amount of tonicity agent will vary, depending on the particular agent to be added and the type of composition. In general, however, the compositions will have a tonicity agent in an amount sufficient to cause the final composition to have an acceptable osmolality.

Regarding buffers, an appropriate buffer system (such as, for example, sodium phosphate, sodium acetate, sodium citrate, sodium borate or boric acid) may be added to the compositions to prevent pH drift under storage conditions. The particular concentration will vary, depending on the agent employed. Preferably, however, the buffer will be chosen to maintain a target pH within the range of pH 3-7 or a range as described herein.

Regarding a surfactant, various surfactants useful in conventional formulations may be employed. Exemplary surfactants include amphoteric surfactants, alkyl amine oxides, anionic surfactants, sodium xylenesulfonate, alone or in combination with Cremophor® EL, polyoxyl 20 ceto stearyl ether, polyoxyl 40 hydrogenated castor oil, polyoxyl 23 lauryl ether and poloxamer 407.

Regarding viscosity building agents, such agents may be added to compositions of embodiments of the present invention to increase the viscosity of the carrier. Examples of viscosity enhancing agents include, but are not limited to: synthetic silicates, polysaccharides, such as hyaluronic acid and its salts, chondroitin sulfate and its salts, dextrans, various polymers of the cellulose family; vinyl polymers; and acrylic acid polymers. For example, the composition may exhibit a viscosity of 1 to 400,000 centipoises (“cps”), and in various embodiments is at least about 50 cps, at least about 100 cps, at least about 200 cps, at least about 500 cps, at least about 1000 cps, or at least about 10,000 cps.

The composition can also include other therapeutic agents such as other antimicrobial agents, anti-inflammatory agents, antihistamines, decongestants, antibiotics, and/or moisturizing agents known in the art.

In certain embodiments, the hypohalous acid composition and the silver additive are formulated and applied as a single composition. In other embodiments, the hypohalous acid composition and the silver additive are formulated individually, and can be applied simultaneous or sequentially. For example, the hypohalous acid composition can be applied as a liquid or hydrogel formulation, and the silver additive can be formulated as a coating or additive on a wound dressing or medical device. The term “simultaneously” as used herein, means that the hypohalous acid composition and the silver additive are applied with a time separation of no more than about 60 minutes, such as no more than 30 minutes, no more than 20 minutes, no more than 10 minutes, no more than 5 minutes, or no more than 1 minute. The term “sequentially” as used herein means that the hypohalous acid composition and the silver additive are applied with a time separation of more than about 60 minutes. For example, the time between the sequential administration of the hypohalous acid composition and the silver additive can be more than 60 minutes, more than 2 hours, more than 5 hours, more than 10 hours, or more than 1 day apart.

In some embodiments, the invention comprises applying hypochlorous acid to a medical device or wound dressing surface having a silver additive coating. The silver additive coating and hypochlorous acid is as described herein. Exemplary devices include a urinary catheter, mucous extraction catheter, suction catheter, umbilical cannula, intrauterine device, intravaginal device, intraintestinal device, endotracheal tube, bronchoscope, endoscope, electrodes, or external prosthesis, as well as others described herein.

In certain embodiments, the combination of a hypohalous acid composition and a silver additive produces a synergistic biocidal effect. As such, the method of the present invention may comprise administration of a hypohalous acid composition and a silver additive, where one or both of the hypohalous composition and the silver additive are administered at a dose below which would be effective alone. Methods for measuring biocidal activity of a composition are known in the art.

In various embodiments, the present invention also provides kits for treating a surface for microbial infection or contamination, comprising a formulation of a hypohalous acid (e.g., hypochlorous acid) composition and a silver additive. The kits may further include materials for wound dressing. Materials for wound dressings are described, for example, in U.S. Pat. Nos. 4,728,323, 7,005,556, and 7,462,752.

### Microbial Infections

The present invention provides methods for treating surfaces affected by microbial infection or colonization.

In certain embodiments, the microbial infection, colonization, or contamination comprises gram positive bacterial infection. Due to structural differences in bacterial cell walls, Gram-positive bacteria generally retain the methyl violet component of Gram's stain after elution with an organic solvent such as ethyl alcohol. Thus, Gram-positive bacteria are bacteria that are stained dark blue or violet by Gram staining. Exemplary Gram-positive microorganisms include, but are not limited to, Staphylococcus aureus, Staphylococci, Streptococci, Enterococci, Carynebacteria, Clostridium (e.g., Clostridium difficile), Listeria and Bacillus (e.g., Bacillus anthracis) species.

In some embodiments, the microbial infection, colonization, or contamination comprises gram negative bacterial infection. A Gram negative bacterium is a bacterium with a cell wall structure that does not retain the methyl violet component of Gram's stain after elution with an organic solvent such as ethyl alcohol. The pink counterstain makes the Gram-negative bacteria appear pink. Gram negative bacteria include, but are not limited to, Escherichia sp. (e.g., E. Coli), Salmonella sp. (e.g., S. typhimurium), Pseudomonas sp. (e.g., P. aeruginosa), Burkholderia sp., Neisseria sp. (e.g., N. meningitides, N. gonorrhoeae), Hemophilus sp. (H. influenzae), Shigella sp. Bactericides sp., Campylobacter sp., Brucella sp., Vibrio sp., Yersinia sp., Helicobacter sp., Calymmatobacterium sp., Legionella sp., Leptospira sp., Borrelia sp., Bordetella sp., Klebsiella sp. (e.g., K. pneumoniae), Treponema sp.; Francisella sp.; Moraxella sp.; Stenotrophomonas sp.; Bdellovibrio sp., Acinetobacter sp., Spirochaetes, Proteus sp. (e.g., Proteus microbilis), Enterobacter, Serratia sp. (e.g., S. plymuthica, S. liquefaciens, S. rubidaea, and S. odoriferae), Gardnerella sp., and any combinations thereof.

In some embodiments, the microbial infection, colonization, or contamination is due to fungi, e.g., yeast, molds. Exemplary fungi and yeast include, but are not limited to, Cryptococcus neoformans, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Aspergillus fumigatus, Aspergillus flavus, Aspergillus clavatus, Cryptococcus neoformans, Cryptococcus laurentii, Cryptococcus albidus, Cryptococcus gattii, Histoplasma capsulatum, Pneumocystis jirovecii (or Pneumocystis carinii), Stachybotrys chartarum, and any combinations thereof.

In other embodiments, the microbial infection, colonization, or contamination involves spores (e.g., bacterial spores or fungal spores). Killing, inactivating, or otherwise reducing the active population of bacterial or fungal spores on surfaces is particularly difficult.

Bacterial spores have a unique chemical composition of spore layers that make them more resistant than vegetative bacteria to the antimicrobial effects of chemical and physical agents. Likewise, the unique chemical composition of fungal spores (e.g., mold spores), makes them more resistant to chemical and physical agents. Bacterial spores can include, for example, Clostridium sp. spores and Bacillus sp. spores. Exemplary bacterial spores include, but are not limited to, spores from Bacillus anthracis, Bacillus cereus, Bacillus subtilis, Bacillus putida, Bacillus pumila, Clostridium tetani, Clostridium Botulinum, and Clostridium difficile. Exemplary fungal spores include, but are not limited to, spores from Aspergillus sp. and Penicillium sp.

Microbial infection or contamination can be present anywhere. In some embodiments, the microbial infection can be present in a subject. For example, the microbial infection can be present on a human or animal tissue or organ. In certain embodiments, the microbial infection is present on a wound, burn, surgical cavity, or bone. In certain embodiments of the invention, the wound needing care is a stage I-IV pressure ulcer, stasis ulcer, diabetic ulcer, post-surgical wound, burn, cut, abrasion, or a minor irritation of the skin.

In various embodiments, the microbial infection or contamination can be present as a biofilm on a hard surface or the surface of a physical object (e.g., medical device). A biofilm is an aggregate of microbes which adhere to each other and/or to a surface, and can form on living or non-living surfaces in various settings. Biofilms are typically less susceptible to conventional antibiotics, antimicrobials, and biocides. Accordingly, the present invention also provides methods for treating a surface having a microbial biofilm.

Many microbes can form biofilms. For example, a Gram-negative bacteria Pseudomonas aeruginosa is known to form biofilms and is an important causative agent of emerging nosocomial infections (also known as hospital-acquired infection such as catheter-associated urinary tract infection). Dental plaque is a biofilm on the surfaces of the teeth and can consist of Gram-negative or Gram-positive bacterial cells (e.g., Streptococcus mutans and Streptococcus sanguis), salivary polymers and bacterial extracellular products. Gram-negative Legionella bacteria are known to grow under certain conditions in biofilms, in which they are protected against disinfectants. Gram-negative Neisseria gonorrhoeae is a human pathogen that has been demonstrated as forming biofilms on glass surfaces and over human cells (e.g., in sexually transmitted infection gonorrhea). Other types of bacteria that form biofilms include, for example, Staphylococcus aureus and Enterococcus sp.

Biofilms are known to be involved in a wide variety of microbial infections in the body of a subject. Infectious processes in which biofilms have been implicated include common problems such as urinary tract infections, catheter infections, middle-ear infections, formation of dental plaque, gingivitis, coating contact lenses, endocarditis, and infections in cystic fibrosis. Biofilms can also be formed on the inert surfaces of medical devices such as catheters, prosthetic cardiac valves and intrauterine devices. Microbial biofilms can also impair wound healing (e.g., cutaneous wound healing) and reduce topical antibacterial efficiency in healing or treating infected skin wounds.

Biofilms on medical devices can be composed of gram-positive or gram-negative microbes (e.g., bacteria). Bacteria commonly found on the medical devices include the gram-positive Enterococcus faecalis (E. faecalis), Staphylococcus epidermidis (S. epidermidis), Staphylococcus aureus (S. aureus), Streptococcus viridans (St. viridans); and the gram-negative Escherichia coli (E. Coli, Klebsiella pneumoniae (K. pneumoniae), Proteus mirabilis (P. mirabilis) and Pseudomonas aeruginosa (P. aeruginosa). The microbes most commonly found in urinary catheter biofilms are Staphylococcus epidermidis, Enterococcus faecalis, E. Coli, Proteus mirabilis, Pseudomonas aeruginosa and Klebsiella pneumoniae. In the case of vascular catheters, Staphylococcus aureus and Staphylococcus epidermidis account for almost 70-80% of all infectious organisms. Candida albicans accounts for about 10-15% of catheter infections. Gram-negative bacilli account for almost 60-70%, enterococci for about 25% and Candida albicans for about 10% of cases of urinary tract infections. Other bacteria that form biofilms include Klebsiella oxytoca, Staphylococcus saprophyticus, Providencia stuartii, Citrobacter freundii and Serratia marcescens.

The present invention also provides methods for preventing the formation of microbial biofilms. For example, the present invention provides methods for preventing formation of a microbial biofilm on a surface (e.g., a surface of a medical device or equipment).

Examples of a hard surface that can be protected and coated using the present invention include, but are not limited to, a tubing, contact lens, dental prosthesis, orthodontic device, surgical instrument, dental instrument, medical examination surface, bathroom surface, dental water line, fabric, or bandage or tissue dressing.

Examples of medical devices that can be protected and coated using the present invention include, but are not limited to, include a urinary catheter, mucous extraction catheter, suction catheter, umbilical cannula, intrauterine device, intravaginal device, intraintestinal device, endotracheal tube, bronchoscope, endoscope, electrodes, or external prosthesis. A medical device can also include any device which can be placed at the insertion or implantation site such as the skin near the insertion or implantation site, and which can include at least one surface which is susceptible to colonization by microbe-embedded biofilms. Medical devices can also include surfaces of equipment in operating rooms, emergency rooms, hospital rooms, clinics, and bathrooms.

The methods and compositions as disclosed herein can also be used in various fields as where antiseptic treatment or disinfection of materials is required, for example, surface disinfection, including for use in bioremediation, such as industry settings, including cleaning of heating and cooling systems, such as HVAC systems.

Accordingly, the present invention meets a substantial public health need. The present invention is effective for preventing and controlling the microbial infections, colonizations, or contaminations on various surfaces and materials, not only for the care and management of wounds, but for disinfecting hard surfaces such as medical or dental equipment, preserving and decontaminating food products, water treatment, as well as other industrial and agricultural applications.

### EXAMPLES

### Example 1

A biofilm test was conducted using hypochlorous acid solutions containing either 200 ppm or 600 ppm AFC. A solution containing a 1:1 combination of silver chelate (30 ppm) and hypochlorous acid (600 ppm) which yielded a final concentration of 200 ppm AFC was also tested.

Specifically, the biocidal effects of the solutions were tested using an in vitro wound biofilm model as described in Werthen et al. (2009), Molnlycke Healthcare. Briefly, a collagen solution was prepared in simulated wound fluid and the collagen was allowed to polymerize at 35° C. for one hour. The collagen matrix was subsequently innoculated with approximately 10⁴ colony forming units (CFU) of Pseudomonas aeruginosa and incubated for twenty-four hours. The hypochlorous acid solutions were added to each matrix and incubated at 35° C. for twenty minutes. The tubes were centrifuged to separate the test solutions from the matrices. Subsequently, each matrix was digested with collagenase, washed and any surviving cells were recovered.

Results demonstrated that the 200 ppm hypochlorous acid solution resulted in one log reduction of P. aeruginosa colony forming units (CFU). The 600 ppm hypochlorous acid solution resulted in a seven log reduction in CFU. Surprisingly, the solution containing a 1:1 combination of silver (30 ppm) and hypochlorous acid (600 ppm) also yielded a seven log reduction in CFU even though the final AFC content of the composition was only 200 ppm. Accordingly, the composition comprising a combination of hypochlorous acid and silver (with a final AFC content of 200 ppm) exhibited a six logarithmic unit increase in biocidal activity compared to a solution containing 200 ppm of hypochlorous acid only.

### Example 2

A biofilm test was conducted to compare the biocidal effects of various hypochlorous acid solutions with or without silver additives, either in form of potassium silver citrate or silver chelate. Specifically, the effects of the solutions on P. aeruginosa biofilm were assessed using an in vitro wound biofilm model as described above. The solutions tested included hypochlorous acid solution with 200 ppm, 400 ppm, 500 ppm, or 600 ppm AFC. Solutions comprising hypochlorous acid and silver were also tested, which included a solution including 300 ppm of AFC and 15 ppm of silver as well as a solution including 300 ppm of AFC and 50 ppm of silver.

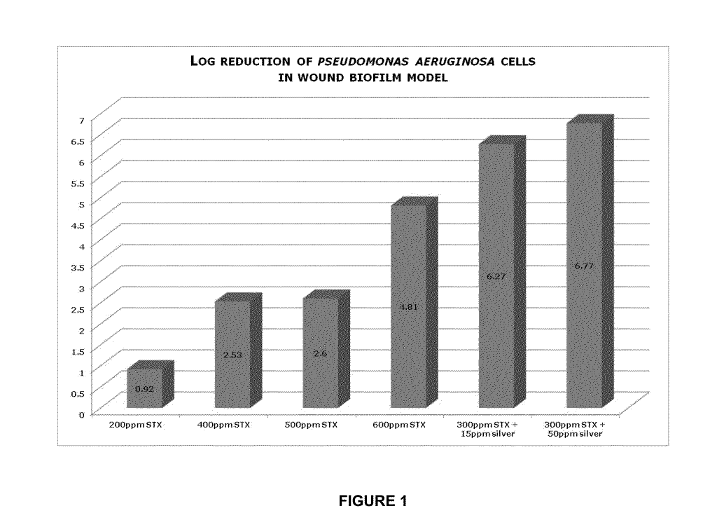

Results as shown in FIG. 1 demonstrate that addition of silver to hypochlorous acid significantly enhanced the biocidal performance of the solution in a biofilm state compared to solutions including hypochlorous acid alone. It is believed that the combination of silver and hypochlorous acid generates silver chloride and other species with enhanced and sustained antimicrobial activities.

What is claimed is:

1. A method for treating a surface characterized by microbial infection or colonization, comprising applying a hypochlorous acid (HOCl) composition and a silver additive to the surface, thereby reducing the microbial infection or colonization.

2. The method of claim 1, wherein the surface is a hard surface.

3. The method of claim 2, wherein the surface is a tubing, contact lens, dental prosthesis, orthodontic device, surgical instrument, dental instrument, medical examination surface, bathroom surface, dental water line, fabric, or bandage or tissue dressing.

4. The method of claim 1, wherein the surface is a medical device.

5. The method of claim 4, wherein the medical device is a urinary catheter, mucous extraction catheter, suction catheter, umbilical cannula, intrauterine device, intravaginal device, intraintestinal device, endotracheal tube, bronchoscope, endoscope, electrodes, or external prosthesis.

6. The method of claim 1, wherein the surface is a human or animal tissue or organ.

7. The method of claim 6, wherein the surface is a wound or burn.

8. The method of any one of claims 1 to 7, wherein the surface is characterized by bacterial biofilm formation.

9. The method of any one of claims 1 to 8, wherein the HOCl and silver additive are applied in a single composition.

10. The method of any one of claims 1 to 8, wherein the HOCl and silver additive are applied sequentially.

11. The method of claim 10, wherein the HOCl is applied as a liquid or hydrogel formulation, and the silver additive is a coating or additive on a wound dressing or medical device.

12. The method of any one of claims 1 to 11, wherein the HOCl composition is produced by electrolysis of saline.

13. The method of claim 12, wherein the HOCl composition has a pH of from about 3.5 to about 7.

14. The method of any one of claims 1 to 13, wherein the HOCl composition has an available chlorine concentration of from about 20 ppm to about 2000 ppm.

15. The method of any one of claims 1 to 14, wherein the silver additive is one or more of elemental silver, silver salt, silver chelate, or ionic silver.

16. The method of claim 15, wherein the silver additive is silver nitrate, silver acetate, silver benzoate, silver citrate, silver carbonate, silver iodate, silver iodide, silver lactate, silver laurate, silver oxide, silver palmitate, and silver sulfadiazine.

17. The method of any one of claims 1 to 16, wherein the silver additive is applied as a solution or ointment having a concentration of at least about 5 ppm.

18. The method of claim 17, wherein the silver additive is applied as a solution or ointment having a concentration of about 5 ppm to about 200 ppm.

19. The method of any one of claims 1 to 18, wherein the bacterial infection comprises a gram positive bacterial infection or gram negative bacterial infection.

20. The method of claim 19, wherein the bacterial infection comprises one or more of E. Coli, Salmonella sp., Shigella sp., Pseudomonas sp., Moraxella sp., Helicobacter sp., Legionella sp., Acinetobacter sp., Neisseria sp., Hemophilus influenzae, Klebsiella pneumoniae, Proteus mirabilis, Bacillus anthracis, Clostridium sp. and Enterobacter sp.

21. The method of claim 20, wherein the surface is infected or colonized with Pseudomonas aeruginosa biofilm.

22. A kit for treating a surface for microbial contamination or infection, comprising: a formulation of a hypochlorous acid (HOCl) composition, and a silver additive.

23. The kit of claim 22, wherein the HOCl composition is a liquid or hydrogel formulation.

24. The kit of claim 22 or 23, wherein the silver additive is a solution or ointment.

25. The kit of claim 22 or 23, wherein the silver additive is a coating or composition integral to a wound dressing or medical device.

26. The kit of claim 25, wherein the medical device is a urinary catheter, mucous extraction catheter, suction catheter, umbilical cannula, intrauterine device, intravaginal device, intraintestinal device, endotracheal tube, bronchoscope, endoscope, electrodes, or external prosthesis.

27. The kit of any one of claims 22 to 26, wherein the HOCl composition is produced by electrolysis of saline.

28. The kit of claim 27, wherein the HOCl composition has a pH of from about 3.5 to about 7.

29. The kit of any one of claims 22 to 28, wherein the HOCl composition has an available chlorine concentration of from about 20 to about 2000 ppm.

30. The kit of any one of claims 22 to 29, wherein the silver additive is silver nitrate, silver acetate, silver benzoate, silver carbonate, silver iodate, silver iodide, silver lactate, silver laurate, silver oxide, silver palmitate, and silver sulfadiazine.

31. The kit of claim 30, wherein the silver additive concentration is at least about 5 ppm.

32. The kit of claim 31, wherein the silver additive concentration is about 5 ppm to about 200 ppm.

33. The kit of any one of claims 22 to 32, wherein the kit further comprises materials for a wound dressing.
